(19) United States
(12) Patent Application Publication
(10) Pub. No.: US 2022/0248950 A1
(43) Pub. Date: Aug. 11, 2022

(54) OPHTHALMIC DEVICE

(71) Applicant: Topcon Corporation, Itabashi-ku, Tokyo (JP)

(72) Inventors: Zhenguo WANG, Ridgewood, NJ (US); Zaixing MAO, Harrison, NJ (US); Jongsik KIM, Fort Lee, NJ (US); Bin CAO, Wayne, NJ (US); Makoto FUJINO, Tokyo (JP); Kazuhiro OOMORI, Tokyo (JP); Eiichi YANAGI, Tokyo (JP); Ying DONG, Warren, NJ (US); Song MEI, Franklin Park, NJ (US); Kinpui CHAN, Ridgewood, NJ (US)

(73) Assignee: Topcon Corporation, Itabashi-ku, Tokyo (JP)

(21) Appl. No.: 17/596,330

(22) PCT Filed: Jun. 10, 2020
(86) PCT No.: PCT/JP2020/022809

§ 371 Date: Dec. 8, 2021

Related U.S. Application Data
(60) Provisional application No. 62/859,231, filed on Jun. 10, 2019.

(51) Int. Cl. A61B 3/10; A61B 3/14; A61B 3/00

(52) U.S. Cl. CPC A61B 3/1005; A61B 3/0008; A61B 3/14

(57) ABSTRACT

An ophthalmologic apparatus includes: an objective lens 18 configured to face a subject's eye E; an illumination optical system 1c configured to irradiate the subject's eye E with illumination light L1; a measurement optical system 1 b configured to take an interference image of corneal reflection light R1, which is a reflection of the illumination light L1, through the objective lens 18; an observation optical system 1a configured to image an anterior segment of the subject's eye E through the objective lens 18; a control unit 2 configured to process information on imaging by the measurement optical system 1b and the observation optical system 1 a; and the control unit 2 configured to simultaneously output, to a single output unit 3, tear film information calculated from the interference image by the measurement optical system 1 b, and information on the anterior segment E imaged by the observation optical system 1a.

### TECHNICAL FIELD

The present disclosure relates to an ophthalmologic apparatus, and mainly to an ophthalmologic apparatus that examines states of an anterior segment and tear fluid film of a subject's eye.

### BACKGROUND ART

There has been known an ophthalmologic apparatus that irradiates a cornea of a subject's eye with illumination light, and observes a state of an anterior segment and an interference image formed by a tear film of the cornea of the subject's eye to make a diagnosis of dry eye, for example.

For example, Patent Document 1 describes an ophthalmologic apparatus that guides light for illuminating an subject's eye to a predetermined point of an oil layer, which is the outermost layer, of the tears of the subject's eye, receives the light reflected from the predetermined point of the oil layer, receives the interference pattern of the interference between the light reflected from the front and back surfaces of the oil layer, and calculates a value indicating the symptom of dry eye based on an output signal.

### CITATION LIST

### Patent Document

PATENT DOCUMENT 1: Japanese Unexamined Patent Publication No. 2000-287930

### SUMMARY OF THE INVENTION

### Technical Problem

A easily readable and understandable report on a result of examining an interference image needs to be output so that an ophthalmologist or other practitioner accurately and easily determines, for example, a subtle dry eye symptom of a subject. However, the typical ophthalmologic apparatus described in Patent Document 1 fails to integrally output examination result information on the examination of the interference image with consistency, which may cause difficulty for the ophthalmologist or other practitioner in accurately and easily determining the conditions of the subject.

The present disclosure was made to solve the problems. It is an objective of the present disclosure to provide an ophthalmologic apparatus including an output unit that outputs a report on a result of examining an interference image. Here, the “result of examining” includes not only an examination result obtained after the end of an examination time but also an examination result during an examination time, such as a live image or progress information, obtained until the middle of the examination time.

### Solution to the Problems

An ophthalmologic apparatus of an aspect of the present disclosure includes: an objective lens configured to face a subject's eye; an illumination optical system configured to irradiate the subject's eye with illumination light; a measurement optical system configured to take an interference image of corneal reflection light, which is a reflection of the illumination light, through the objective lens; an observation optical system configured to image an anterior segment of the subject's eye through the objective lens; a control unit configured to process information on imaging by the measurement optical system and the observation optical system; and the control unit configured to simultaneously output, to a single output unit, tear film information calculated from the interference image by the measurement optical system, and information on the anterior segment imaged by the observation optical system.

### Advantage of the Invention

The present disclosure provides an ophthalmologic apparatus including an output means that outputs a report on a result of examining an interference image.

### BRIEF DESCRIPTION OF THE DRAWINGS

FIG. 1 is a schematic diagram showing a configuration of an ophthalmologic apparatus according to an embodiment of the present disclosure.

FIG. 2 is a schematic view of a display screen showing examination conditions on an output unit of the ophthalmologic apparatus according to the embodiment of the present disclosure.

FIG. 3 is a schematic view of a display screen showing a report on an examination result on the output unit of the ophthalmologic apparatus according to the embodiment of the present disclosure.

FIG. 4 is a schematic view showing a variation of the display screen showing examination conditions on the output unit of the ophthalmologic apparatus according to the embodiment of the present disclosure.

FIG. 5 is a schematic view showing a variation of the display screen showing a report on an examination result on the output unit of the ophthalmologic apparatus according to the embodiment of the present disclosure.

FIG. 6 is a schematic view showing a variation of the display screen on the output unit of the ophthalmologic apparatus according to the embodiment of the present disclosure.

FIG. 7 is a schematic view showing another variation of the display screen on the output unit of the ophthalmologic apparatus according to the embodiment of the present disclosure.

FIG. 8 is a schematic view showing further another variation of the display screen on the output unit of the ophthalmologic apparatus according to the embodiment of the present disclosure.

FIG. 9 is a schematic view showing yet another variation of the display screen on the output unit of the ophthalmologic apparatus according to the embodiment of the present disclosure.

### DESCRIPTION OF EMBODIMENT

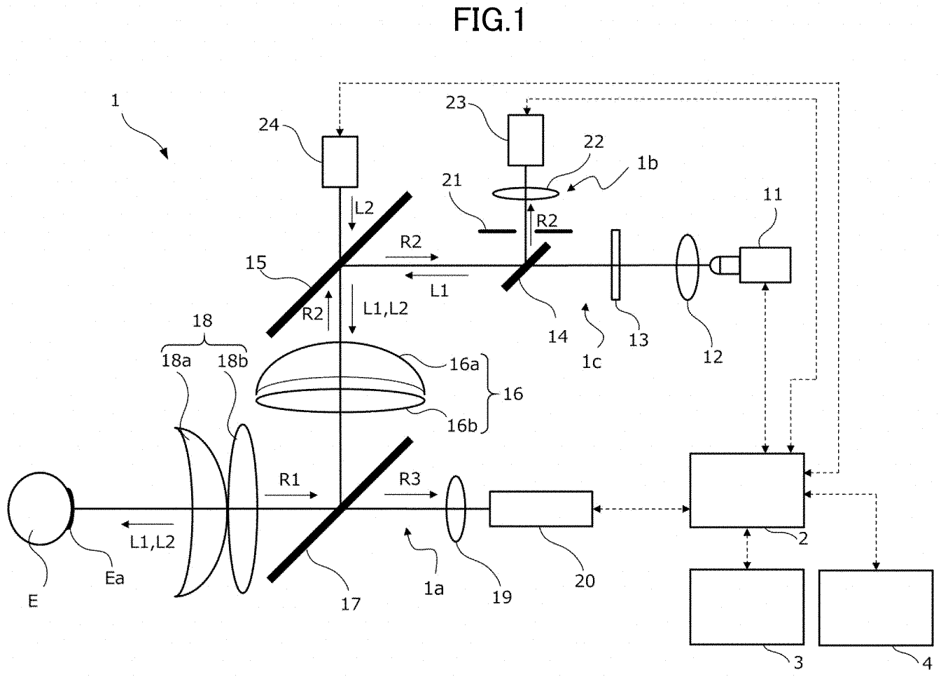

FIG. 1 is a schematic view showing a configuration of an ophthalmologic apparatus 1 according to an embodiment of the present disclosure. The optical system of the ophthalmologic apparatus 1 includes an anterior segment observation optical system 1a, a corneal measurement optical system 1b, and an illumination optical system 1c.

The anterior segment observation optical system 1a includes a first lens group 18 of the present disclosure. The anterior segment observation optical system 1a includes a third half mirror 17, an imaging lens 19, and an anterior segment camera 20 that are arranged along the direction of an optical axis of the first lens group 18.

The first lens group 18 is a so-called objective lens. In the present embodiment, the objective lens (first lens group 18) includes a plurality of lenses (18a, 18b), but the objective lens may include a single lens only. This first lens group 18 allows the corneal surface of a cornea Ea of the subject's eye E to be irradiated with illumination light L1 emitted from the illumination optical system 1c, which will be described later, via the third half mirror 17. Corneal reflection light R1, which is the reflection of the illumination light from the corneal surface, enters the first lens group 18. This corneal reflection light R1 enters the third half mirror 17 from the first lens group 18.

The third half mirror 17 reflects part of the illumination light L1 incident from the illumination optical system 1c toward the first lens group 18. The third half mirror 17 allows part (R3) of the corneal reflection light R1 incident from the first lens group 18 to pass therethrough and exit therefrom toward the imaging lens 19, and reflects further part (R2) of the corneal reflection light R1 toward a second lens group 16, which will be described later.

The imaging lens 19 allows the corneal reflection light R3 incident from the third half mirror 17 to pass therethrough and exit therefrom toward the anterior segment camera 20. The anterior segment camera 20 includes a complementary metal oxide semiconductor (CMOS) or charge coupled device (CCD) imaging element, and takes an image of the corneal reflection light R3 incident from the imaging lens 19 to output an imaging signal of an observation image of an anterior segment of the subject's eye E (hereinafter referred to as an “anterior segment observation image”) to a control unit 2. The observation image of the anterior segment may be output as an observation image obtained by observing the states of the cornea, conjunctiva, and tears using a fluorescent dye such as fluorescein staining.

The illumination optical system forms an optical path branching from the anterior segment observation optical system via the third half mirror 17.

The illumination optical system 1c includes an illumination light source 11. The illumination optical system 1c further includes a lens 12, a filter 13, a first half mirror 14, a second half mirror 15, and the second lens group 16 which are arranged on an optical path of illumination light L1 emitted from the illumination light source 11. The illumination optical system 1c shares the third half mirror 17 and the first lens group 18 with the anterior segment observation optical system 1a. The illumination optical system 1c forms an optical path branching from the anterior segment observation optical system 1a via the third half mirror 17.

The illumination light source 11 emits light. The illumination light source 11 may be, for example, a light emitting diode (LED) light source or halogen lamp which emits white light, and emits white light as the illumination light L1 toward the lens 12. Alternatively, an LED having a different wavelength, a laser light source, or a combination of them may also be used. The lens 12 allows the illumination light L1 incident from the illumination light source 11 to exit therefrom toward the filter 13. The filter 13 adjusts the light intensity and/or wavelength distribution of the illumination light L1 incident from the lens 12, and allows the illumination light L1 thus adjusted to exit therefrom toward the first half mirror 14.

The first half mirror 14 may allow part of the illumination light L1 incident from the filter 13 to pass therethrough and exit therefrom toward the second half mirror 15. The first half mirror 14 reflects part of the corneal reflection light R2 incident from the second lens group 16, which will be described later, via the second half mirror 15 toward the corneal measurement optical system 1b, which will be described later.

In this manner, the corneal surface of the cornea Ea is irradiated with, through the first lens group 18, the illumination light L1 emitted from the illumination light source 11 and passing through the lens 12 and the third half mirror 17. As a result, the corneal reflection light R1, which is the reflection of the illumination light L1 from the corneal surface, enters the first lens group 18.

The corneal measurement optical system 1b forms an optical path branching from the illumination optical system 1c via the first half mirror 14. The corneal measurement optical system 1b shares the components from the first lens group 18 to the first half mirror 14 with the illumination optical system 1c, and also includes a diaphragm 21, a lens 22, and an interference image capturing camera 23.

The diaphragm 21 and the lens 22 allow the corneal reflection light R2 incident from the first half mirror 14 to exit therefrom toward the interference image capturing camera 23.

The interference image capturing camera 23 includes a CMOS or CCD imaging element, and takes an image of the corneal reflection light R2 incident from the lens 22 to output an imaging signal of a corneal reflection image to the control unit 2.

A fixation lamp 24 is a light source that fixes the position of the subject's eye E by guiding the subject's gaze for accurate observation and photographing of the state of the subject's eye E. A light emitting diode (LED) light source, or a halogen lamp can be used as the fixation lamp 24. The light L2 emitted from the fixation lamp 24 passes through the second half mirror 15 and the second lens group 16, is reflected by the third half mirror 17, and enters the subject's eye E through the first lens group 18.

The control unit 2 is electrically connected to an output unit 3, a database unit 4, the illumination light source 11, the anterior segment camera 20, the interference image capturing camera 23, and the fixation lamp 24.

The control unit 2 detects, based on the image data (i.e., a corneal reflection image) of the corneal reflection light R2 input from the interference image capturing camera 23, the wavelength characteristics of the interference image at each position of the corneal reflection image. Accordingly, the control unit 2 calculates the thickness of the tear film at each position on the surface of the cornea Ea. The control unit 2 detects an abnormality such as a foreign body like dust using a technique such as edge detection.

The control unit 2 includes a storage unit. The control unit 2 obtains two-dimensional (2D) dynamic information on the tear film using the interference image capturing camera 23 and stores the dynamic information in the storage unit. The control unit 2 then generates examination result information from the interference image stored in the storage unit based on information obtained at a plurality of times. Accordingly, the control unit 2 extracts a tear film breakup region (dry eye region) and a tear film breakup time. In addition, the control unit 2 displays, on the output unit 3, information on the detected thickness of the tear film, information on a map of the thickness distribution, and information on the position of an abnormal region (dry spot). The tear fluid film herein refers to an oil layer (lipid layer), an aqueous layer, and a mucinous layer, or a combination of these layers.

The control unit 2 further outputs, to the output unit 3, a live observation image of the anterior segment in real time based on an imaging signal input from the anterior segment camera 20. Accordingly, real-time images of, for example, the tear film, the cornea, and/or the anterior segment are captured. Although not shown, blood vessels of the retina may be observed using a slit lamp to capture an image of the retina.

The control unit 2 allows the output unit 3 to display information on the tear film, for example. The control unit 2 also displays, on the output unit 3, the tear film information calculated from the interference image captured by the interference image capturing camera 23 and the information on the anterior segment captured by the anterior segment camera 20 after superimposing the tear film information and the information on the anterior segment. Alternatively, these two images obtained by the interference image capturing camera 23 and the anterior segment camera 20 may be displayed side by side.

The output unit 3 is a device capable of outputting an image and/or information transmitted from the control unit 2. The output unit 3 may be, for example, a display device such as a liquid crystal display or a CRT device. The output unit 3 may be a PC, a tablet PC, a smartphone, a head-mounted display, and smart glasses that are attached or mounted with a display; a projector; or a printer. The information displayed on the output unit 3 is operatable by an input through an operation unit (not shown). The operation unit may be, for example, an input device such as a keyboard or a mouse, or a touch panel integral with a display device such as a liquid crystal display. The output unit 3 may be configured to perform display simultaneously using a plurality of, for example, two display devices.

The database unit 4 stores information such as the thickness of the tear film, the thickness of the lipid layer, the tear film breakup region, and the tear film breakup time obtained from a large number of subjects. These information is associated with information such as age and/or sex, and stored as a standard data of general (average) values. In addition, information such as the thickness of a tear film, the thickness of a lipid layer, a tear film breakup region, and a tear film breakup time that are specific to a certain disease is stored in association with the disease. Note that the database unit 4 may store various information in association with identification markers such as IDs.

The control unit 2 refers to and automatically compares the information in the database unit 4 using an observation result and a measurement result transmitted from the anterior segment observation optical system 1a and the corneal measurement optical system 1b, respectively, to determine an examination result and the conditions of a patient. The database unit 4 may be connected to the control unit 2 via a network such as the Internet, or may be integral with the control unit 2.

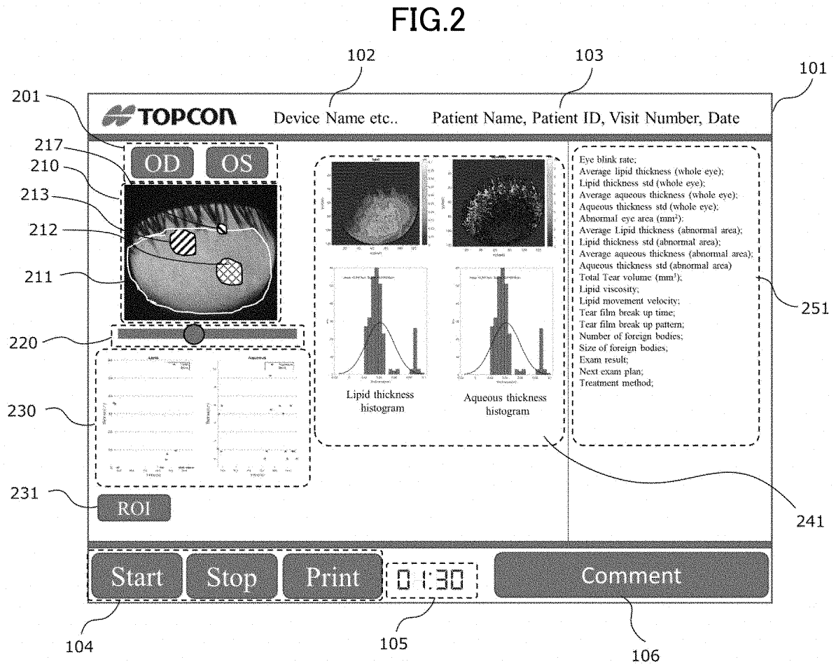

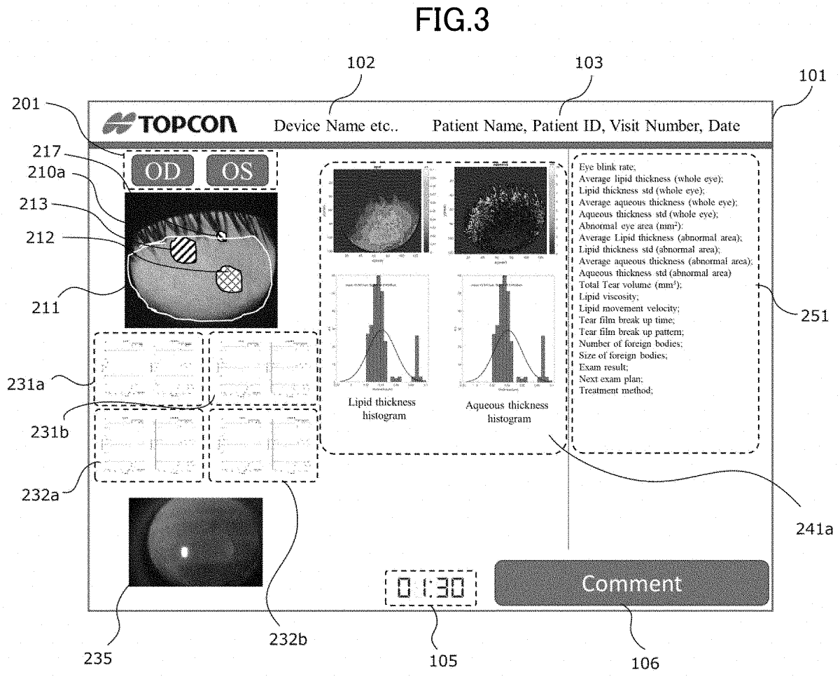

Next, with reference to FIGS. 2 and 3, a display screen will be described using an example where the output unit 3 is a display device.

FIG. 2 is a display screen 101 of the output unit 3 displaying examination conditions.

Displayed on the top of FIG. 2 are a company logo (Company Logo), device information 102 (Device Information), and patient information 103 (Patient Information).

Displayed on the upper left of FIG. 2 is an eye selection button 201 for selecting an eye to be displayed, with which an oculus dexter (OD, i.e., the right eye) or an oculus sinister (OS, i.e., the left eye) is selectable. A user such as an ophthalmologist selects the eye to be displayed by clicking the displayed button “OD” or “OS” using a mouse, for example. In addition, both the “OD” and “OS” buttons may be clicked to display the examination results of both eyes side by side for comparison.

Displayed on the middle left of FIG. 2 is a live tear film image 210 (Live tear film image). In the live tear film image 210, an eye region 211 (Eye region) to be diagnosed may be segmented, and foreign bodies 217 (Foreign bodies) may be highlighted. In addition, the control unit 2 detects and displays an abnormal tear film region 212 (Abnormal tear film region) and/or a suspicious tear film breakup region 213 (Suspicious tear film breakup region). Displayed under the live tear film image 210 is a slide bar 220 (Slide bar). The user moves the slide bar 220 after the end of the examination to reproduce information as of any time based on the information stored in the storage unit.

Displayed on the lower left of FIG. 2 is a graph area 230 related to a region of interest (ROI) and including graphs showing the thicknesses of the lipid and aqueous layers and the time (Lipid and Aqueous thickness vs Time (ROI)), and the change rates of the thicknesses of the lipid and aqueous layers (Thickness changing rate (ROI)). Displayed under the graphs is an “ROI” button for the user to select the region of interest. The user presses an “ROI” button 231 to select the region of interest (ROI) within the live tear film image 210.

Displayed on the upper center of FIG. 2 is a histogram display 241 showing the thicknesses of the lipid and aqueous layers and the histograms of the respective thicknesses. Displayed in this section are (2D) thickness maps of the lipid and aqueous layers of the whole eye to be observed according to the live tear film image 210. Being stored in the storage unit, the thicknesses of these two layers are reproduced at a time selected using the slide bar 220 after the end of measurement. Displayed under the maps are the histograms of the thicknesses of the lipid and aqueous layers. The left is the histogram of the thickness of the lipid layer, whereas the right is the histogram of the thickness of the aqueous layer.

Displayed on the right of FIG. 2 is a parameter information display 251 showing the parameter information on the tear film (Tear film Parameters). Examples of such information include an eye blink rate (“Eye blink rate”), the average thickness of the lipid layer of the whole eye (“Average lipid thickness (whole eye)”), the standard thickness of the lipid layer of the whole eye (“Lipid thickness std (whole eye)”), the average thickness of the aqueous layer of the whole eye (“Average aqueous thickness (whole eye)”), the standard thickness of the aqueous of the whole eye (“Aqueous thickness std (whole eye)”), the area of an abnormal tear film region (“Abnormal eye area (mm²)”), the average thickness of the lipid layer in the abnormal region (“Average Lipid thickness (Abnormal area)”), the standard thickness of the lipid layer in the abnormal region (“Lipid thickness std (Abnormal area)”), the average thickness of the aqueous layer in the abnormal region (“Average aqueous thickness (Abnormal area)”), the standard thickness of the aqueous layer in the abnormal region (“Aqueous thickness std (abnormal area)”), a total tear volume (“Total Tear volume (mm³)”), the viscosity of the lipid layer (“Lipid viscosity”), the moving speed of the lipid layer (“Lipid movement velocity”), a tear film breakup time (“Tear film break up time”), a tear film breakup pattern (“Tear film break up pattern”), the number of foreign bodies (“Number of foreign bodies”), the sizes of the foreign bodies (“Size of foreign bodies”), an examination result (“Exam result”), a next examination plan (“Next exam plan”), and a treatment method (“Treatment method”). Here, “std” represents the standard deviation (Standard Deviation).

Displayed on the bottom of FIG. 2 are control buttons (Control button section) 104, an imaging time display 105 (Imaging time), and a comment display 106 of the comments of a doctor or other practitioner (Comment section). The control buttons 104 include three buttons of “Start”, “Stop”, and “Print”. “Start” is pressed to start the recording of the observed conditions and store images or other information in the storage unit. “Stop” is pressed to stop the recording. “Print” is pressed to output the display screen and/or necessary information to an external output device such as a printer. The imaging time display 105 indicates the time elapsed since the press of “Start”, that is, the start of the recording. The comment display 106 is an area in which a doctor or other practitioner inputs any comments through an input unit.

Next, FIG. 3 shows a screen of the output unit 3 displaying a report on an examination result. Only the display sections changed from FIG. 2 will be described below.

Displayed on the middle left of FIG. 3 are graphs 231a showing the thicknesses of the lipid and aqueous layers in regions of interest (ROIs) and the time (Lipid and Aqueous thickness vs time (multiple ROIs)), and the change rates of the thicknesses of the lipid and aqueous layers in the ROIs (Thickness changing rate (multiple ROIs)). Found on the right are graphs 231b showing the average thicknesses of the lipid and aqueous layers of the whole eye (whole eye) and the time (Average lipid and Aqueous thickness VS time (whole eye)), and the change rates of the thicknesses of the lipid and aqueous layers of the whole eye (Thickness changing rate (whole eye)). Displayed under the graphs 231a are graphs 232a showing a comparison between the measured thicknesses of the lipid and aqueous layers and general data stored in the database unit 4 (Average lipid and aqueous thickness vs Normative database). Displayed on the right are graphs 232b showing the volume of the aqueous layer and the time (Aqueous volume VS Time), and the change rate of the volume of the aqueous layer (Aqueous volume changing rate). Displayed on the lower left of FIG. 3 is a fluorescence image 235 (Fluorescence image) measured by a fluorescence method. The display may include reference images and a plurality of fluorescence images registered in a two-dimensional thickness map.

Displayed on the upper center of FIG. 3 is a histogram display 241a showing the thicknesses of the lipid and aqueous layers and the histograms of the respective thicknesses. Displayed in this section are (2D) thickness maps of the lipid and aqueous layers of the whole eye to be observed according to a tear film image 210a at a selected time. Displayed under the maps are the histograms of the thicknesses of the lipid and aqueous layers. The left is the histogram of the thickness of the lipid layer, whereas the right is the histogram of the thickness of the aqueous layer.

In this manner, the control unit 2 displays, on the output unit 3, the display images shown in FIGS. 2 and 3 to be readable at a glance so that the conditions of examining an interference image and a report on the examination result are easily readable and understandable for the user such as an ophthalmologist.

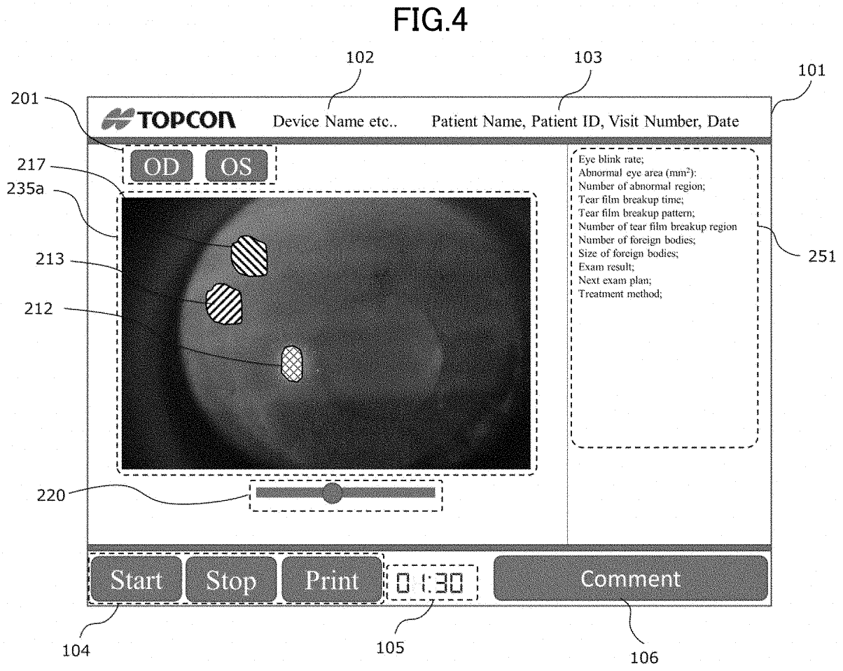

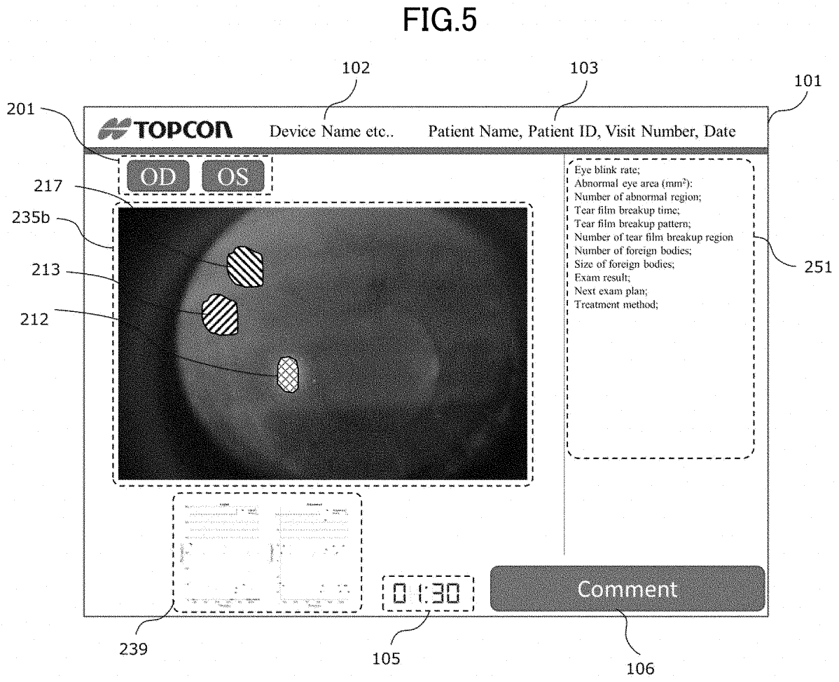

Next, with reference to FIGS. 4 and 5, a variation of the display screen shown in FIGS. 2 and 3 will be described using an example where the output unit 3 is a display device.

FIG. 4 is a display screen 101 of the output unit 3 displaying an examination result.

Displayed on the top of FIG. 4 are a company logo (Company Logo), device information 102 (Device Information), and patient information 103 (Patient Information).

Displayed on the upper left of FIG. 4 is an eye selection button 201 (Eye Choose) for selecting an eye to be displayed, with which an oculus dexter (OD, i.e., the right eye) or an oculus sinister (OS, i.e., the left eye) is selectable. A user such as an ophthalmologist selects the eye to be displayed by clicking the displayed button “OD” or “OS” using a mouse, for example. In addition, both the “OD” and “OS” buttons may be clicked to display the examination results of both eyes side by side for comparison.

Displayed around the center of FIG. 4 is a live fluorescence image 235a in a live fluorescein staining test. In the fluorescence image 235a in the live fluorescein staining test, the eye region (Eye region) and foreign bodies 217 (Foreign bodies) may be highlighted, and an abnormal tear film region 212 (Abnormal tear film region) and/or a suspicious tear film breakup region 213 (Suspicious tear film breakup region) may be detected and displayed. Displayed under the fluorescence image 235a is a slide bar 220 (Slide bar). The user moves the slide bar 220 after the end of the examination to reproduce the fluorescence image as of any time stored in the storage unit.

Displayed on the right of FIG. 4 is a parameter information display 251 showing the parameter information on the tear film (Tear film Parameters). Examples of such information include an eye blink rate (“Eye blink rate”), the area of an abnormal tear film region (“Abnormal eye area (mm²)”), the number of abnormal regions (“Number of abnormal region”), a tear film breakup time (“Tear film breakup time”), a tear film breakup pattern (“Tear film breakup pattern”), the number of tear film breakup regions (“Number of tear film breakup region”), the number of foreign bodies (“Number of foreign bodies”), the sizes of the foreign bodies (“Size of foreign bodies”), the examination result (“Exam result”), a next examination plan (“Next exam plan”), and a treatment method (“Treatment method”).

Displayed on the bottom of FIG. 4 are control buttons 104 (Control button section), an imaging time display 105 (Imaging time), and a comment display 106 of the comments of the user such as an ophthalmologist (Comment section). The control buttons 104 include three buttons of “Start”, “Stop”, and “Print”. “Start” is pressed to start the recording of the observed conditions and store images or other information in the storage unit. “Stop” is pressed to stop the recording. “Print” is pressed to output the display screen and/or necessary information to an external output device such as a printer. The imaging time display 105 indicates the time elapsed since the press of “Start”, that is, the start of the recording. The comment display 106 is an area in which the user such as an ophthalmologist inputs any comments through an input unit.

Next, FIG. 5 shows a screen of the output unit 3 displaying a report on an examination result. Only the display sections changed from FIG. 4 will be described below.

Displayed on the lower left of FIG. 5 is a graph region 239 showing the areas of abnormal regions (1, 2, . . . ) and time (Abnormal area (1, 2 . . . ) VS time), and the change rates of the areas of the abnormal regions (Abnormal area changing rate) that are calculated and displayed by the control unit 2. The abnormal regions may include here some or all of an abnormal tear film region (Abnormal tear film region), a suspicious tear film breakup region (Suspicious tear film breakup region), a tear film breakup region (Tear film breakup region), or foreign bodies (Foreign bodies). The changes of theses over time are shown.

In this manner, the control unit 2 displays, on the output unit 3, the display images shown in FIGS. 4 and 5 so that a report on a result of examining an interference image is easily readable and understandable for the user such as an ophthalmologist.

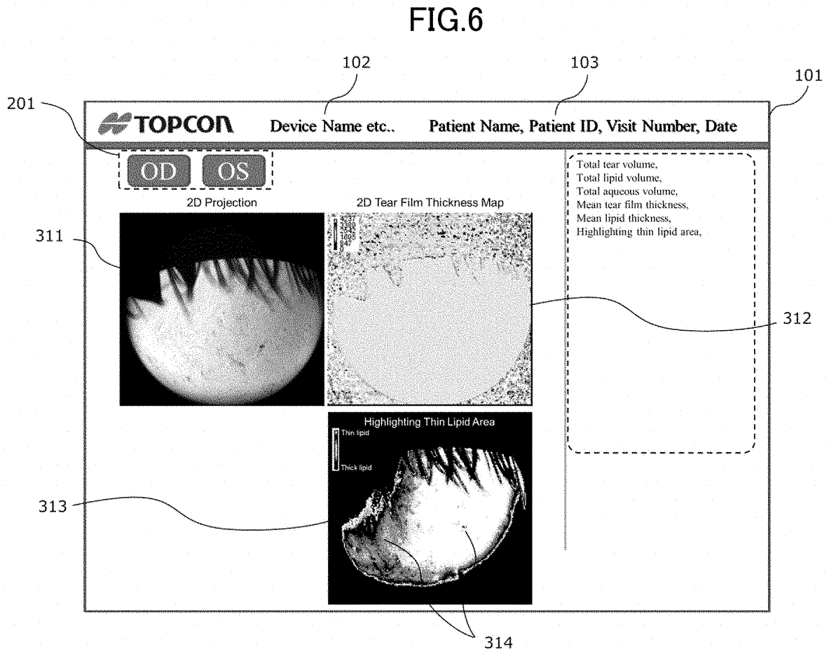

FIGS. 6 to 9 are schematic views showing other variations of the display screen on the output unit of the ophthalmologic apparatus according to the embodiment of the present disclosure.

FIG. 6 shows visualized two-dimensional thickness maps of the tear film with a region of the lipid layer with a smaller thickness highlighted. The thickness maps are calculated by the control unit 2 from a hyper-spectral image captured using a hyper-spectral camera as the anterior segment camera 20. The region of the lipid layer with a smaller thickness calculated from the two-dimensional thickness map of the tear film may be further emphasized in the simulated color map to assist a doctor or other practitioner in making a determination on clinical evaluation.

Displayed on the upper left of FIG. 6 is a projection image 311 generated by the hyper-spectral camera. Displayed around the center is a two-dimensional thickness map 312 of the tear film calculated by the control unit 2 from the hyper-spectral image. Displayed on the bottom is a two-dimensional projection image 313 generated from the hyper-spectral image. In this image, the region of the lipid layer with a smaller thickness is displayed in a warm color as a highlighting 314.

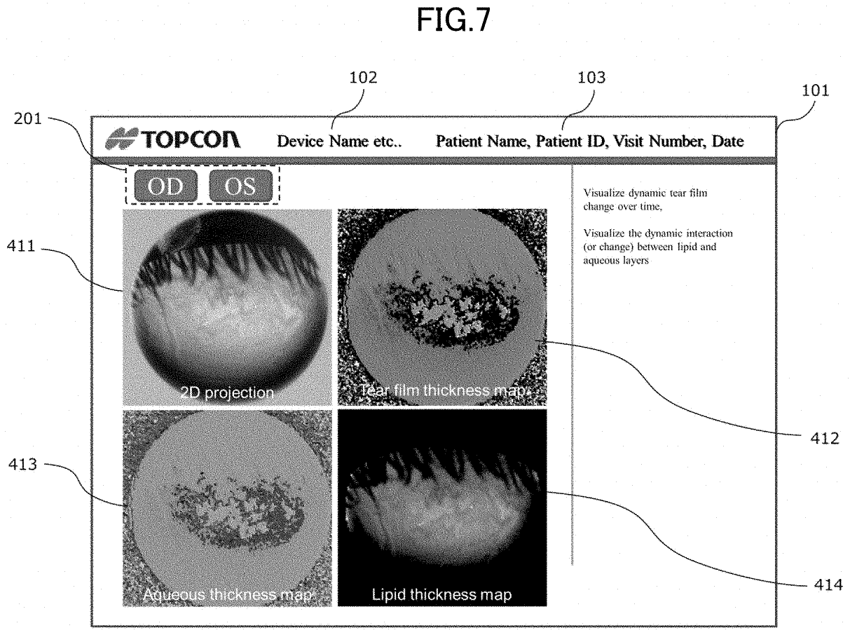

FIG. 7 shows, on one screen, a two-dimensional projection image 411 (i.e., the “2D projection” image generated by hyper-spectral imaging), a two-dimensional thickness map 412 of the tear film (i.e., a 2D “tear film thickness map”), a thickness map 413 of the aqueous layer (i.e., a 2D “aqueous thickness map”), and a thickness map 414 of the lipid layer (i.e., a 2D “lipid thickness map”). These images are useful to clarify the dynamic properties of the tear film, particularly the lipid and aqueous layers.

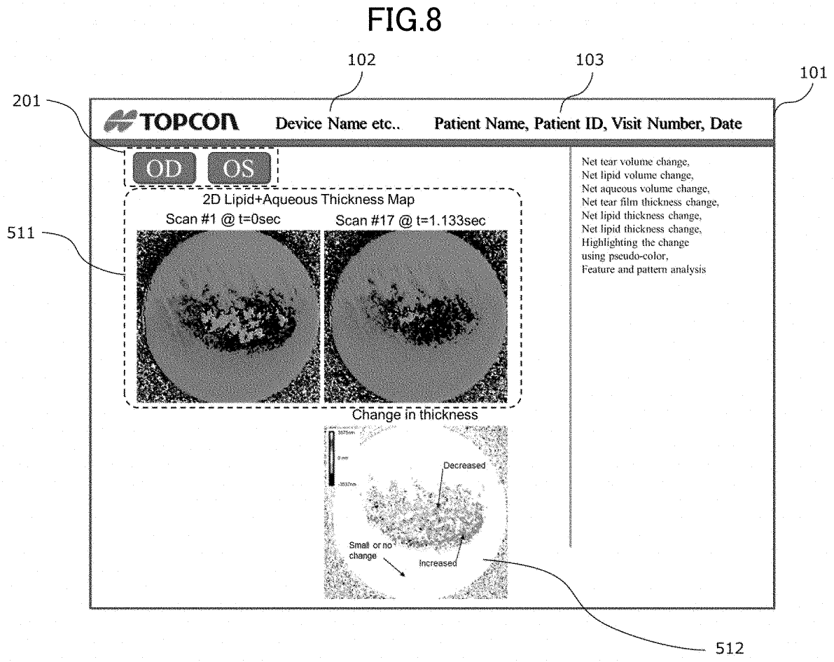

FIG. 8 shows an image of the net change in the thickness of the tear film. The film thickness in the cross section is measured twice at different time points and the net change is calculated by the control unit 2. The obtained value is then converted into intensity and displayed by the control unit 2 for visual examination. Upper images 511 are the two-dimensional thickness maps of the aqueous layer (i.e., the tear film) at two different times, Scan #1 (at 0 seconds) and Scan #17 (after 1.133 seconds). A lower image 512 visualizes the relative change in the thickness of the tear film over time, and is calculated by the control unit 2 based on the data on the upper images, Scan #1 and Scan #17.

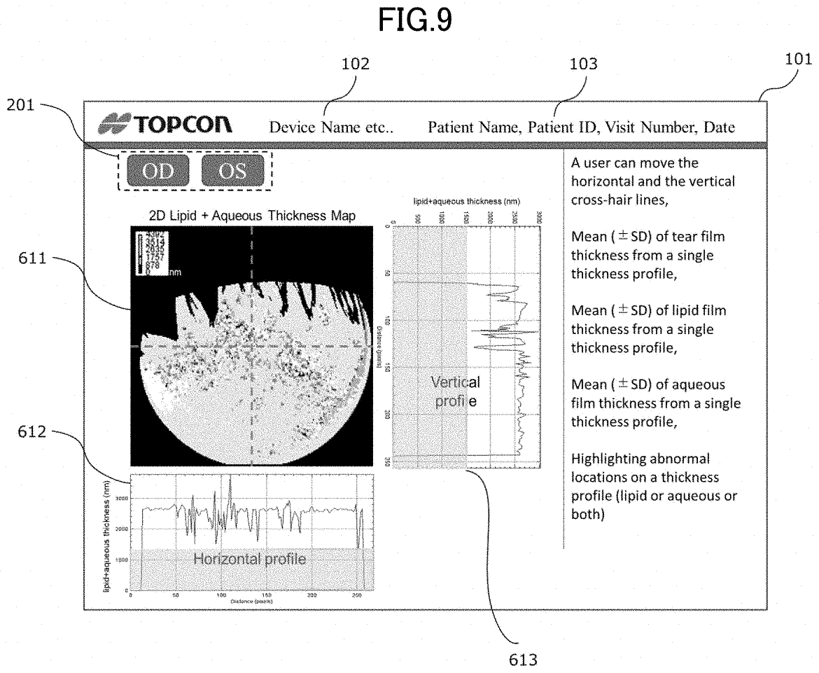

FIG. 9 shows an image 611 that is a two-dimensional image generated from a hyper-spectral image and indicating the thickness map of the lipid and aqueous layers. Displayed here are film thickness profiles in the cross section along virtual lines (e.g., horizontal and vertical lines). A graph 612 is the horizontal profile, whereas a graph 613 is the vertical profile. The control unit 2 applies the range of the abnormal conditions derived based on the clinical data stored in the database unit 4 to these film thickness profiles to detect and highlight the abnormal regions on the image.

In the ophthalmologic apparatus according to the present disclosure, the output unit 3, which is a display device, may switchably display one of the screens shown in FIGS. 2 to 9, or may freely select and display the contents to be displayed on the screen. In addition, the contents displayed in each figure may be all listed on the screen and the numerical values of keywords may be displayed, or only the keywords necessary for the user such as an ophthalmologist may be displayed.

As described above, the ophthalmologic apparatus according to the present disclosure outputs an easily readable report on a result of examining an interference image. Accordingly, not only a skilled ophthalmologist but also an ophthalmologist with little examination experience easily recognizes a subtle dry eye symptom.

### DESCRIPTION OF REFERENCE CHARACTERS

1: Ophthalmologic Apparatus
1a: Anterior Segment Observation Optical System
1b: Corneal Measurement Optical System
1c: Illumination Optical System
2: Control Unit
3: Output Unit
4: Database Unit
11: Illumination Light Source
12: Lens
13: Filter
14: First Half Mirror
15: Second Half Mirror
16: Second Lens Group
17: Third Half Mirror
18: First Lens Group
19: Imaging Lens
20: Anterior Segment Camera
21: Diaphragm
22: Lens
23: Interference Image Capturing Camera
24: Fixation Lamp
101: Display Screen
102: Device Information
103: Patient Information
104: Control Button
105: Imaging Time Display
106: Comment Display
201: Eye Selection Button
210: Live Tear Film Image
210a: Tear Film Image
211: Region
212: Tear Film Region
213: Tear Film Breakup Region
217: Foreign Body
220: Slide Bar
230: Graph Area
231: ROI Button
231a, 231b: Graph
232a, 232b: Graph
235, 235a: Fluorescence image
239: Graph Region
241, 241a: Histogram Display
251: Parameter Information Display
311: Projection Image
312: Thickness Map of Tear Film
313: Projection Image
314: Highlighting
411: Two-Dimensional Projection Image
412: Two-Dimensional Thickness Map of Tear Film
413: Thickness Map of Aqueous Layer
414: Thickness Map of Lipid Layer
511: Image
512: Image
611: Image
612: Graph
613: Graph
E: Subject's Eye
Ea: Cornea

1. An ophthalmologic apparatus comprising:
an objective lens configured to face a subject's eye;
an illumination optical system configured to irradiate the subject's eye with illumination light;
a measurement optical system configured to take an interference image of corneal reflection light, which is a reflection of the illumination light, through the objective lens;
an observation optical system configured to image an anterior segment of the subject's eye through the objective lens; and
a control unit configured to process information on imaging by the measurement optical system and the observation optical system,
the control unit being configured to simultaneously output, to a single output unit, tear film information calculated from the interference image by the measurement optical system, and information on the anterior segment imaged by the observation optical system.

2. The ophthalmologic apparatus of claim 1, wherein
the control unit simultaneously outputs, to the output unit, the tear film information calculated from the interference image by the measurement optical system, and the information on the anterior segment imaged by the observation optical system after superimposing the tear film information and the information on the anterior segment.

3. The ophthalmologic apparatus of claim 1, wherein
the tear film information indicates a tear film breakup region.

4. The ophthalmologic apparatus of claim 1, wherein
the tear film information indicates a dry eye region.

5. The ophthalmologic apparatus of claim 1, wherein
the tear film information indicates a region including a foreign body.

6. The ophthalmologic apparatus of claim 1, wherein
the control unit outputs examination result information to the output unit.

7. The ophthalmologic apparatus of claim 6, wherein
the control unit generates the examination result information using information on a thickness of a tear film, a thickness of a lipid layer, a tear film breakup region, and a tear film breakup time that are obtained from a large number of subjects and stored in a database unit, and the tear film information and/or the information on the anterior segment imaged by the observation optical system.

8. The ophthalmologic apparatus of claim 1, wherein
the control unit outputs parameter information on the tear film to the output unit.

9. The ophthalmologic apparatus of claim 7, wherein
the parameter information on the tear film includes at least one of an average thickness of the lipid layer, an average thickness of the tear film, a standard thickness of the tear film, an area of an abnormal region, an average thickness of the lipid layer in the abnormal region, a standard thickness of the lipid layer in the abnormal region, an average thickness of the tear film in the abnormal region, a total volume of tears in the eye, a viscosity of the lipid layer, a moving speed of the lipid layer, a tear film breakup time, or a tear film breakup pattern.

10. The ophthalmologic apparatus of claim 1, wherein
the control unit outputs, to the output unit, information including at least one of a blink rate, a number of foreign bodies, sizes of foreign bodies, an examination result, a next examination plan, or a treatment method.

11. The ophthalmologic apparatus of claim 1, wherein
the output unit is a display device.
